(19) United States
(12) Patent Application Publication
(10) Pub. No.: US 2026/0069280 A1
(43) Pub. Date: Mar. 12, 2026

(54) TUBULAR BRAIDED IMPLANTABLE ENDOVASCULAR EMBOLIZATION DEVICE

(71) Applicant: DePuy Synthes Products, Inc., Raynham, MA (US)

(72) Inventors: Daniel SOLAUN, Miami, FL (US); Lacey GOROCHOW, Miami, FL (US); Pedro D. PEDROSO, Parkland, FL (US); Patrick BROUWER, Haarlem (NL)

(73) Assignee: DePuy Synthes Products, Inc., Raynham, MA (US)

(21) Appl. No.: 18/828,558

(22) Filed: Sep. 9, 2024

(51) Int. Cl. A61B 17/12; A61B 17/00; A61M 25/00

(52) U.S. Cl. CPC A61B 17/12172; A61B 17/12113; A61B 2017/00526; A61B 2017/00867; A61B 2017/1205; A61M 25/0068

(57) ABSTRACT

An endovascular embolization system including a braided implantable device having: a tubular braid pre-formed as a default shape into multiple distinct sections including: an intermediate section interposed directly between a stabilizing section and a sealing section; the intermediate section having a stiffness greater than that of the stabilizing section; and a proximal section. Each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the distal section, the intermediate section, and the proximal section.

### FIELD

The present disclosure relates to an endovascular medical treatment device, and more particularly, to a tubular braided implantable endovascular embolization device to disrupt blood flow at the target site. By way of non-limiting example, the tubular braided implantable endovascular embolization device may be used in the treatment of an aneurysm to divert (i.e., impede) blood flow or to restrict blood flow in a vessel (e.g., in the brain or peripheral vasculature).

### BACKGROUND

An aneurysm stretches out thereby thinning a section of the wall of the artery. Cranial aneurysms may be difficult to treat due to their proximity to critical brain tissues. Conventional solutions have included endovascular treatment whereby an internal volume of the aneurysm sac is surgically removed or excluded from arterial blood pressure and flow via an endovascular intrasaccular device. Current alternatives to endovascular intrasaccular devices or other surgical approaches include endovascularly delivered treatment devices that fill the sac (i.e., dome) of the aneurysm with embolic material or block the entrance (i.e., neck) of the aneurysm. Both approaches attempt to prevent or divert blood flow into the aneurysm. By filling an aneurysm sac, the embolic material clots the blood, creating a thrombotic mass within the aneurysm. Whereas, treating the aneurysm neck, blood flow into the entrance of the aneurysm is impeded, inducing venous stasis in the aneurysm and facilitating a natural formation of a thrombotic mass within the aneurysm.

Current intravascularly delivered implantable embolization devices typically utilize multiple devices (e.g., embolic coils) to either fill the sac or treat the entrance (i.e., neck) of the aneurysm. Naturally formed thrombotic masses created by treating the entrance with embolic coils may result in improved healing compared to aneurysm masses packed with embolic coils because naturally formed thrombotic masses can reduce the likelihood of distention from arterial walls and facilitate reintegration into the original parent vessel shape along the neck plane. However, embolic coils delivered to the neck of the aneurysm can potentially have the adverse effect of impeding the flow of blood in the adjoining blood vessel, particularly if the entrance is overpacked. Conversely, if the entrance is insufficiently packed, recanalization of blood flow may persist into the aneurysm. Treating certain aneurysm morphology (e.g., wide neck, bifurcation, etc.) may require ancillary devices (e.g., stents or balloons) to support the coil mass and obtain the desired packing density. Once implanted, the coils cannot easily be retracted or repositioned. Furthermore, aneurysms treated with multiple coils over time often recanalize or compact resulting from poor coiling, lack of coverage across the aneurysm neck, blood flow, and/or relatively large aneurysm size.

Alternatives to embolic coils are being explored, for example a tubular braided implant as disclosed in U.S. Pat. Nos. 10,653,425; 10,751,066; 11,278,292; 11,413,046; and 11,583,282, each of which is incorporated herein by reference in their entirety. Tubular braided implants have the potential to easily, accurately, and safely treat an aneurysm or other arterio-venous malformation in a parent vessel without blocking flow into perforator vessels communicating with the parent vessel. Compared to embolic coils, however, tubular braided implants are a newer technology, and there is therefore capacity for improved geometries, configurations, delivery systems, optimization of disruption of blood flow, etc. Regarding the geometry, it is desirable to design the tubular braided implant to minimize risk of damage to the vessel wall. This is a factor in all vasculature treatment procedures, but particularly significant during treatment of an aneurysm in which the vessel wall is inherently thin. Several factors contribute to optimizing disruption of blood flow. During delivery through the microcatheter, conventional tubular braided implants may undesirably twist in configuration relative to the axial/longitudinal axis extending therethrough. In a twisted orientation the efficiency of the implanted conventional tubular braided device to disrupt blood flow to the aneurysm is compromised. Despite most likely being unsuccessful attempts may be made to untwist the conventional tubular braided implant by torquing the delivery wire while the implanted conventional tubular braided device has exited from the catheter (i.e., while implanted at the target site in the vessel of the patient). If not successfully untwisted while implanted, then the twisted conventional tubular braided implant must be fully withdrawn from the microcatheter. Once outside the body, the twisted conventional tubular braided implant may be manipulated by hand to its original untwisted orientation prior to reattempting delivery of the same device. Otherwise following withdraw of the twisted conventional tubular braided implant from the body a new tubular braided implantable device may be delivered through the microcatheter to the target site. Efficiency of diversion of blood flow may also be optimized by preventing or minimizing probability of migration over time of the tubular braided implanted device (i.e., future recanalization).

It is therefore desirable to develop an improved single (e.g., one piece, unitary) tubular braided implantable endovascular embolization device that addresses all these factors.

### SUMMARY

An aspect of the present disclosure relates to an improved tubular braided implantable endovascular embolization device comprising a stabilizing section apposing the aneurysm wall thereby anchored in place while working in tandem with the intermediate section imposing a force against and stabilizing at the aneurysm neck the sealing section minimizing migration distally over time (i.e., reducing risk of future recanalization).

While another aspect of the present disclosure relates to an improved tubular braided implantable endovascular embolization device with multiple distinct sections of varying stiffness (i.e., rigidity) to minimize risk of damage to the aneurysm wall, maximize compressibility and positioning, and optimize disruption of blood flow. Preferably, of the multiple distinct sections the stabilizing section has a lowest stiffness (i.e., rigidity), the intermediate section having increased stiffness or rigidity relative to the stabilizing section, and the sealing section exhibiting a highest stiffness (i.e., rigidity) compared to that of the intermediate section.

Still another aspect of the present disclosure relates to an improved tubular braided implantable endovascular embolization device including a stabilizing section and a sealing section that when deployed (i.e., upon exiting from the distal end/tip of the microcatheter) each self-expands radially to an implanted size and shape dependent on the anatomy (e.g., size and shape) of the aneurysm in which it is implanted, while and intermediate section disposed therebetween has a smaller outer diameter relative to either of the stabilizing or sealing section while in the radially self-expanded state.

While another aspect of the present disclosure is directed to an improved tubular braided implantable endovascular embolization device that at all times, and in particular, during delivery through the delivery device (e.g., microcatheter)) to the target site, is preferably unrestricted 360 degrees freely rotatable relative to the pushing member (e.g., delivery wire or tube) minimizing twisting in a longitudinal/axial direction thereby optimizing radial expansion and efficiency of diversion of blood flow when implanted at the target site in its non-twisted configuration.

Another aspect of the present disclosure relates to an improved tubular braided implantable endovascular embolization device unrestricted 360 degrees freely rotatable relative to the pushing member (e.g., delivery wire or tube) minimizing, or preventing altogether, twisting imposed by torque friction during delivery through the microcatheter and resulting built-up energy therefore decreasing the track force permitting use of smaller size microcatheters.

Still another aspect of the present disclosure relates to an improved tubular braided implantable endovascular embolization device that when the stabilizing section is implanted at the target site (e.g., in the sac/dome of the aneurysm) substantially centers the microcatheter off the aneurysm wall.

In yet still another aspect the present disclosure relates to an improved tubular braided implantable endovascular embolization device wherein the intermediate section acts as a hinge permitting off angle positioning relative to a longitudinal axis of the sealing section at the neck of the aneurysm relative to that of the stabilizing section while anchored in place in the sac/dome of the aneurysm, such aspect being particularly well suited for treatment of off angle aneurysms.

Another aspect of the present disclosure relates to an improved tubular braided implantable endovascular embolization device including a multi-layer sealing section optimizing disruption (e.g., diversion or occlusion) of blood flow passable though open space regions defined in the multi-layer sealing section, while maintaining maximum compressibility and positioning of the stabilizing section formed from only a single braided layer.

While still yet another aspect of the present disclosure is directed to an improved tubular braided implantable endovascular embolization device with substantially all (e.g., ≥approximately 95%) of the sealing section disposed in the aneurysm (e.g., in the sac/dome including the neck) with minimal (e.g., ≤approximately 5%) protrusion of the sealing section into the parent vessel.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices of the present disclosure, by way of example only, not by way of limitation.

FIG. 1A is a side view of an example of the tubular braided implantable endovascular embolization device in accordance with the present disclosure having multiple distinct sections including: a non-inverted distal section, a stabilizing section, an intermediate section, a sealing section, and a non-inverted proximal section;

FIG. 1B is a longitudinal/axial cross-sectional view along line 1(B)-1(B) through the tubular braided implantable endovascular embolization device of FIG. 1A; wherein the curved arrow denotes unrestricted 360 degrees free rotation of the tubular braided implantable endovascular embolization device relative to a delivery wire;

FIG. 1C is a schematic representation along the longitudinal/axial axis of the tubular braided implantable endovascular embolization device of FIG. 1A depicting distal, intermediate, and proximal securement members disposed about the respective non-inverted distal, intermediate and non-inverted proximal sections each having a single braided layer;

FIG. 1D is a schematic representation along the longitudinal/axial axis of another example of the tubular braided implantable endovascular embolization device wherein the sealing section includes two nested braided layers for heightened stiffness and rigidity, while the stabilizing section includes only a single braided layer to maintain maximum compressibility and positioning at the target site;

FIGS. 2A-2C depict sequential stages of deployment of the tubular braided implantable endovascular embolization device of FIG. 1A in the vasculature during the treatment of an aneurysm;

FIG. 3A-3C depict an enlarged view of the aneurysm during sequential stages of deployment of the tubular braided implantable endovascular embolization device of FIG. 1A;

FIG. 4A is a side view of another example of the tubular braided implantable endovascular embolization device in accordance with the present disclosure having multiple distinct sections including: a non-inverted distal section, a stabilizing section, an intermediate section, a sealing section, and a non-inverted proximal section; and a coil secured to the non-inverted distal section of the tubular braided implantable endovascular embolization device acts as a bumper preventing damage to the vessel during positioning;

FIG. 4B is a schematic representation along the longitudinal/axial axis of the tubular braided implantable endovascular embolization device of FIG. 4A depicting distal, intermediate, and proximal securement members disposed about the respective non-inverted distal, intermediate and non-inverted proximal sections each having a single braided layer;

FIGS. 5A-5G depicting sequential stages of deployment of the tubular braided implantable endovascular embolization device of FIG. 4A during treatment of an aneurysm;

FIG. 6 is a flow chart of the method of using a multi-section tubular braided implantable endovascular embolization device in accordance with the present disclosure wherein optional steps are described within dashed-line boxes;

FIG. 7 is a flow chart of the method of manufacture a multi-section tubular braided implantable endovascular embolization device in accordance with the present disclosure wherein optional steps are described within dashed-line boxes; and

FIGS. 8A-8E depict alternative exemplary multi-section tubular braided implantable endovascular embolization devices in accordance with the present disclosure in which the distal section is inverted inward interiorly of the stabilizing section while the proximal section is inverted inward interiorly of the sealing section; wherein in FIG. 8A the stabilizing and sealing sections are substantially equal in both the x-direction and the y-direction; in FIG. 8B the stabilizing and sealing sections are substantially equal in the x-direction, while the stabilizing section is smaller than the sealing section in the y-direction; in FIG. 8C the stabilizing and sealing sections are substantially equal in the x-direction, while the stabilizing section is larger than the sealing section in the y-direction; in FIG. 8D the stabilizing section is smaller than the sealing section in the x-direction, while the stabilizing sections are substantially equal in the y-direction; and in FIG. 8E the stabilizing section is greater than the sealing section in the x-direction, while the stabilizing and sealing sections are substantially equal in the y-direction.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

One aspect of the present disclosure is directed to a single device for diversion/disruption of blood flow, e.g., diversion of blood flow to an aneurysm or impeding blood flow in a vessel such as in the brain or peripheral vasculature. During treatment of an aneurysm, rather than employing the time-consuming process of packing the sac/dome of the aneurysm with multiple embolization devices (e.g., multiple conventional embolic coils typically of varying size), the delivery and deployment procedure in accordance with the present disclosure is streamlined to only a single assembled tubular braided implantable endovascular embolization device. In addition, the tubular braided implantable endovascular embolization device is self-adjusting to an implanted state having a size and shape dependent on (i.e., conforming to) the anatomy of aneurysms of varying size and shape. Also, the tubular braided implantable endovascular embolization device in accordance with the present disclosure advantageously: (i) minimizes risk of damage to the vessel wall; (ii) maximizes compressibility and positioning at the target site; (iii) maximizes diversion (i.e., impeding) of blood flow away from an aneurysm or restricting (i.e., impeding) blood flow in a vessel (e.g., in the brain or peripheral vasculature); and (iv) minimizes risk of migration in a distal direction over time hence reducing probability of future recanalization.

The tubular braided implantable endovascular embolization device is formed starting with a tubular braid made of a plurality of wires woven into a desired pattern (e.g., full diamond pattern). To maximize compressibility and minimize risk of damage to the vessel wall each of the plurality of wires has an outer diameter preferably ≤approximately 0.001″. The wires are made of a biocompatible memory shape material (e.g., Nitinol—Nickle titanium alloy) pre-formable (e.g., heat set) to have a natural, default, or original shape (i.e., radially expanded state of maximum outer diameter) when free from an externally applied radially compressive or radially constraining force, but radially compressible (i.e., collapsed state having a reduced outer diameter) when subject to the externally applied radially compressive or radially constraining force. The tubular braid preferably forms a right cylinder having an inner passageway 140 extending in an axial/longitudinal direction between a distal free edge at a first open end and a proximal free edge at an opposite second open end.

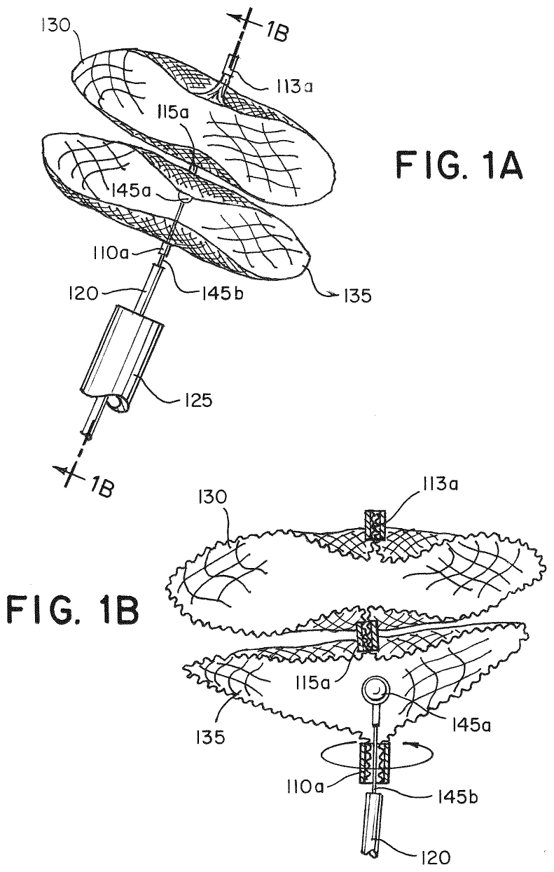

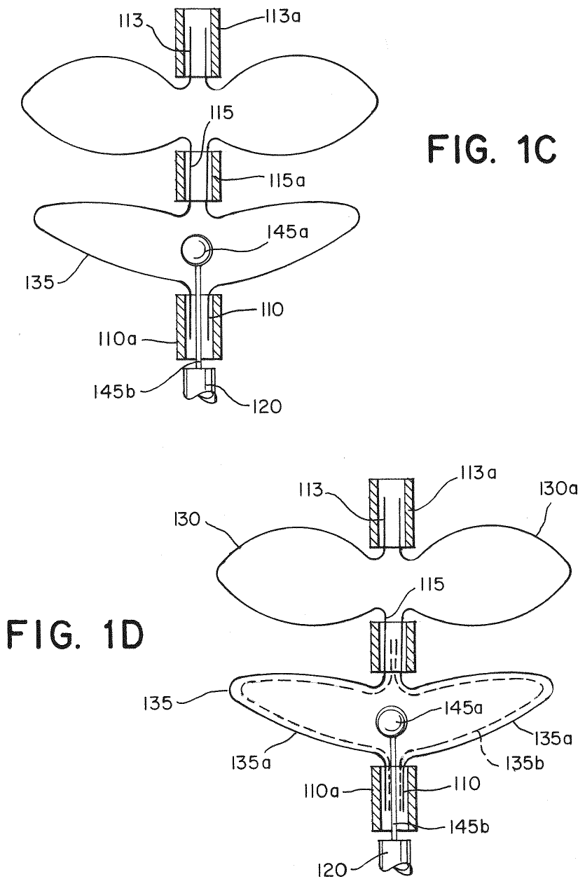

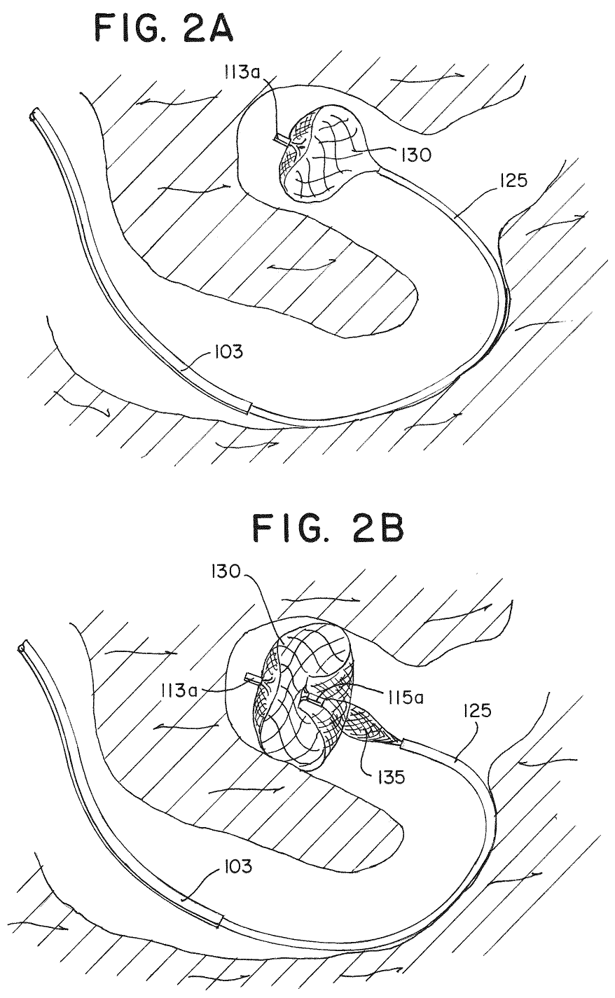

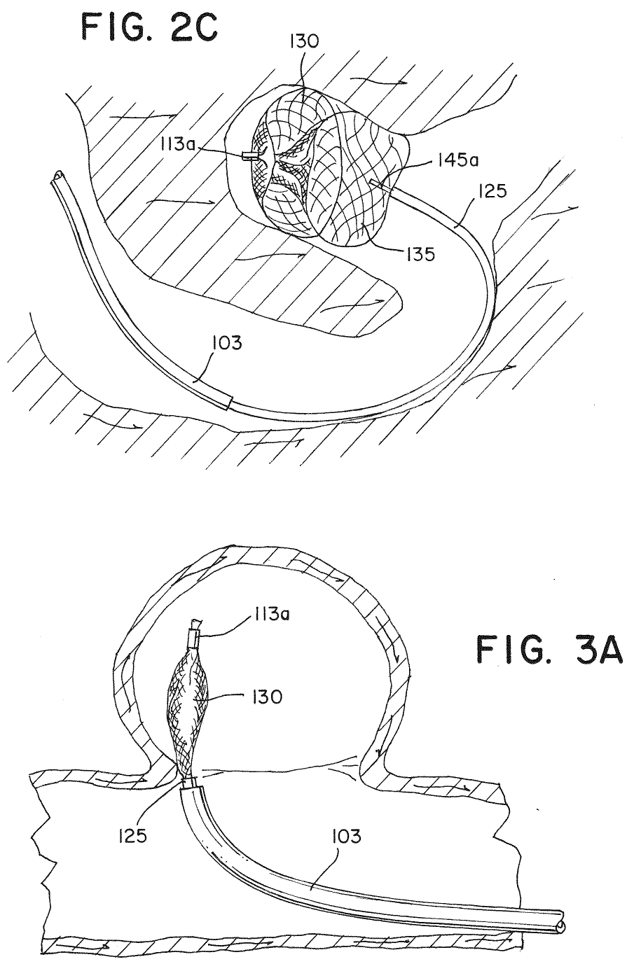

Referring to the example tubular braided implantable endovascular embolization device in FIGS. 1A & 1B, the tubular braid is pre-formed (i.e., heat set) into a natural, default, or original shape of multiple distinct sections each serving different functions during delivery and/or deployment. Starting from a distal most end and extending to the opposite proximal end the tubular braided implantable endovascular embolization device, in series (one directly after the other), includes: a non-inverted distal section 113, a stabilizing section 130, an intermediate section 115, a sealing section 135, and a non-inverted proximal section 110. Non-inverted distal section 113 represents the distal portion of the tubular braided implantable endovascular embolization device and has a reduced outer diameter relative to the stabilizing section 130 while in a radially expanded state, as described in further detail below. A distal securement member 113a (e.g., a distal marker band) is secured about the non-inverted distal section 113. Preferably the smaller outer diameter of the non-inverted distal section 113 is maintained substantially unchanging (i.e., radially non-transitioning) during insertion, delivery through and upon exiting from the microcatheter 125. Next in series in the proximal direction is the stabilizing section 130. The stabilizing section 130 may be pre-formed (e.g., heat set) such that while in a natural, default, or original state has a predetermined radially self-expanding shape of maximum outer diameter. In a natural, default, or original state of maximum outer diameter the stabilizing section 130 may, for example, resemble a torus or inflated inner tire. Other radially self-expanded shapes of the pre-formed stabilizing section 130 are contemplated.

To fulfill the goals of preventing or minimizing risk of damage to the vessel wall (i.e., minimizing stiffness or rigidity) while optimizing compressibility and positioning when deployed at the target site, the stabilizing section 130 is preferably formed by only a single braided layer 130a. In furtherance of these stated goals, individual wires forming the folded over tubular braid preferably each have an outer diameter ≤approximately 0.001″. During delivery to the target site, the stabilizing section 130 is radially collapsed (i.e., reduced in outer diameter) while radially constrained within the lumen of the delivery device (e.g., microcatheter 125). Upon exiting from the distal end/tip of the microcatheter 125, the deployed stabilizing section 130 self-expands radially to an implanted size and shape dependent on the anatomy (e.g., size and shape) of the target site (e.g., sac/dome of the aneurysm or vessel). When deployed, stabilizing section 130 apposes the wall of the aneurysm cavity with sufficient force to remain anchored and stabilized in place. Due to its compressibility, radially and/or longitudinally, the stabilizing section 130 is self-adjusting to the implanted size and shape dependent on the anatomy (e.g., size and/or shape) of the aneurysm or vessel in which it is implanted. When anchored in place in the aneurysm cavity the deployed stabilizing section 130 substantially centers the microcatheter 125 relative thereto assisting during deployment of those remaining sections (e.g., intermediate section 115, sealing section 135, and non-inverted proximal section 110) of the tubular braided implantable endovascular embolization device yet to exit the distal end/tip of the microcatheter 125.

Continuing in a proximal direction, the following section is the intermediate section 115 (i.e., bridge section or stalk section) having a narrow outer diameter that is: (i) smaller relative to that of the pre-formed natural, default, original state radially expanded of maximum outer diameter of either the stabilizing section 130 or the sealing section 135 between which it is directly interposed; and (ii) less than or equal to the inner diameter of the lumen of the microcatheter 125 through which the embolization device is deliverable to the target site. In one example configuration, regardless of the state of any other section of the embolization device at any given time, the narrow diameter of the intermediate section 115 is at all times maintained substantially unchanging in outer diameter (i.e., radially non-transitioning or not radially self-expanding) during introduction into, delivery through, and upon exiting from the microcatheter 125. Such substantially unchanging aforementioned narrow outer diameter of the intermediate section 115 may be realized in several different ways. During manufacture the intermediate section 115 may be pre-formed (e.g., heat set) as a radially non-transitioning (i.e., not radially self-expanding) region having the aforementioned narrow outer diameter. Alternatively, the intermediate section 115 having the aforementioned narrow outer diameter may be repositionable via a mechanical radially constraining device 115a (e.g., intermediate marker band) freely slidable in a longitudinal/axial direction about a pre-formed radially self-expanding region of the tubular braid interposed between the stabilizing section 130 and sealing section 135. During deployment of the embolization device (i.e., while exiting from the distal tip/end of the microcatheter 125), the freely slidable intermediate marker band 115a automatically repositions itself in the longitudinal/axial direction, simultaneously self-adjusting in size the respective stabilizing section 130 and sealing section 135 dependent on the anatomy of the aneurysm. Due to its aforementioned narrow outer diameter, the intermediate section 115 is significantly more difficult to compress (i.e., substantially incompressible) longitudinally/axially and radially providing increased stiffness or rigidity relative to that of the stabilizing section 130 when deployed at the target site. The enhanced stiffness/rigidity of the intermediate section 115 interposed directly between the stabilizing section 130 and the sealing section 135 advantageously provides column strength. While the stabilizing section 130 remains anchored in place, the enhanced stiffness or rigidity of the intermediate section 115 imposes in a proximal direction a force on thereby stabilizing the sealing section 135 at the neck of the aneurysm minimizing risk of migration in a distal direction over time (i.e., future recanalization). Furthermore, intermediate section 115 acts like a hinge allowing independent offset angular positioning of the sealing section 135 relative to the stabilizing section 130 while anchored in place. Such offset angular positioning of the respective sealing section 135 relative to the stabilizing section 130 is particularly well suited for treatment of off angle aneurysms.

Thereafter, in a proximal direction, the next section is the sealing section 135. When the embolization device is being manufactured, the sealing section 135 is pre-formed (i.e., heat set) in a natural, default, original state (i.e., radially expanded state) having a predetermined maximum outer diameter (e.g., resembling an inflated tire inner tube) greater than or equal to the entrance (i.e., neck) of the aneurysm or inner diameter of the blood vessel to be restricted (e.g., occluded). During delivery to the target site, the sealing section 135 while radially collapsed (i.e., reduced in outer diameter) is radially constrained within the lumen of the delivery device (e.g., microcatheter 125). Upon exiting from the distal end/tip of the microcatheter 125, the deployed sealing section 135 self-expands radially to an implanted size and shape dependent on the anatomy (e.g., size and shape) of the aneurysm (including the neck and the sac/dome). The deployed sealing section 135 is stabilized at the neck of the aneurysm via the force imposed by the intermediate section 115 working together with the stabilizing section 130 anchored in place in the sac/dome.

In the treatment of an aneurysm using an implantable embolization device it is desirable to maximize disruption or diversion of blood flow to optimize healing at the site of the neck of the aneurysm. Maximum disruption or diversion of blood flow may be realized by increasing the number of layers (e.g., more than one braided layer) forming the embolization device hence obstructing or impeding passage of blood through the openings defined by the woven braided wires. However, additional layers undesirably hamper compression and positioning of the embolization device in the aneurysm. Both conflicting factors are addressed in the tubular braided implantable endovascular embolization device in accordance with the present disclosure. To promote healing by maximizing disruption or diversion of blood flow the sealing section 135 positionable at the neck of the aneurysm may optionally include multiple layers (e.g., two or more braided layers) nested together one inside the other, while the stabilizing section 130 having only a single layer (e.g., single braided layer) remains unhampered in both compressibility and positioning. This increase in material provided by the nested multiple layers (e.g., two braided layers as illustrated in FIG. 1C) forming the sealing section 135 obstruct or impede (e.g., reducing in size) open space regions defined by the nested multiple layers thereby maximizing disruption without completely/totally prohibiting passage of blood flow therethrough (i.e., more efficient diversion of blood flow away from the aneurysm). In addition to the braided layer 135a formed by the tubular braid, the supplemental obstruction layer(s) 135b comprising the nested multiple layers of the sealing section 135 may include a supplemental braided layer, a supplemental coil, and/or a supplemental suture(s).

Inventive features associated with distinct sections of the tubular braided implantable endovascular embolization device operating together stabilize the sealing section 135 at the neck of the aneurysm minimizing risk of migration in a distal direction over time (i.e., future recanalization). In particular, stabilization at the neck of the aneurysm is maximized by a force imposed thereon in a proximal direction towards the neck of the aneurysm (i.e., in a direction away from the non-inverted distal section 113) by the intermediate section 115 (fostered by its heightened stiffness) while being supported by the stabilizing section 130 anchored in place in combination with the enhanced stiffness of the optional multi-layer (more than one layer) sealing section 135.

Upon exiting from the distal end/tip of the microcatheter 125 into the sac/dome of the aneurysm, the sealing section 135 automatically self-expands radially to its implanted size and shape dependent on the anatomy (e.g., size and shape) of the aneurysm. While visible under imagery (e.g., fluoroscopic imagery), by physically manipulating (e.g., partially withdrawing in a proximal direction and/or advancing in a distal direction) the pushing member 120 (e.g., delivery wire or tube) the sealing section 135 is repositionable to be properly seated at the neck of the aneurysm. When properly seated at the neck of the aneurysm, substantially all (i.e., approximately ≥95%) of the sealing section 135 is disposed within the aneurysm (including both the sac/dome and neck) while only a minimal portion (e.g., ≤5%) protrudes or extends beyond the neck of the aneurysm into the adjacent parent vessel.

The most proximal part of the tubular braided implantable endovascular embolization device is the non-inverted proximal section 110. During manufacture the non-inverted proximal section 110 has a pre-formed radially non-transitioning narrow outer diameter that is: (i) smaller relative to that of the natural, default, original state radially expanded of maximum outer diameter of either the stabilizing section 130 or the sealing section 135; and (ii) less than or equal to the inner diameter of the lumen of the microcatheter 125 through which the embolization device is deliverable through the vasculature to the target site. The narrow outer diameter of each of the respective non-inverted distal section 113, non-inverted proximal section 110, and intermediate section 115 may, but need not necessarily, be substantially equal. Regardless of the state of any other section of the embolization device at any given time, the non-inverted distal section 113 and the non-inverted proximal section 110 preferably are each maintained substantially unchanging in outer diameter. That is, preferably no radial constriction is required of either the non-inverted distal section 113 or the non-inverted proximal section 110 during delivery through the lumen of the microcatheter 125 to the target site nor does either the non-inverted distal section 113 or the non-inverted proximal section 110 undergo any radial expansion upon exiting from the distal end/tip of the microcatheter 125. During manufacture the non-inverted proximal section 110 may be pre-formed (e.g., heat set) as a radially non-transitioning (i.e., not radially self-expanding) region having the aforementioned narrow outer diameter.

Conventional tubular braided implantable endovascular embolization devices are non-rotatably connected to a delivery system (e.g., pushing member). During delivery through the microcatheter to the target site (e.g., aneurysm, blood vessel, etc.) the conventional tubular braided implantable endovascular embolization device non-rotatably attached to the pushing member may undesirably twist about a longitudinal/axial axis. Twisting is caused by advancing (e.g., pushing) the pushing member together with the implant non-rotatably attached thereto through the microcatheter while disposed in the vascular curvature which generates torque on the implant due to friction with the inner wall of the microcatheter. When deployed at the target site, any twisting of the conventional tubular braided implantable endovascular embolization device restricts maximum radial expansion of the implant to its original, natural, default state hampering deployment and implantation. Furthermore, twisting of implant when implanted also diminishes efficiency of diversion or disruption of blood flow away from the aneurysm or through the blood vessel. To prevent or minimize undesirable twisting during delivery the tubular braided implantable endovascular embolization device in accordance with the present disclosure is preferably unrestricted 360 degrees freely rotatably connected to the pushing member 120 (e.g., delivery wire or tube). Therefore, during advancement (i.e., pushing) in a distal direction through the microcatheter 125, the unrestricted 360 degrees free rotation of the tubular braided implantable endovascular embolization device allows automatic self-untwisting about its longitudinal axis during delivery through the microcatheter while remaining attached to the pushing member 120 (e.g., delivery wire or tube). Any residual, remaining or de minimis twisting automatically self-untwists upon the tubular braided implantable endovascular embolization device exiting from the microcatheter all while remaining attached to the pushing member 120 (e.g., delivery wire or tube). FIGS. 1A-1C illustrate an example unrestricted 360 degrees freely rotatable connection of the tubular braided implantable endovascular embolization device relative to the pushing member 120 (e.g., delivery wire or tube). The illustrated example of the unrestricted 360 degrees freely rotatable connection includes a distal interference member 145a (e.g., ball) disposed within an inner space or cavity of the sealing section 135 (i.e., distally of the inner channel of the non-inverted proximal section 110) and having a maximum outer diameter greater than the inner diameter of the non-inverted proximal section 110 preventing passage or escape therethrough in a proximal direction. A tether 145b extending through the longitudinal/axially defined inner channel of the non-inverted proximal section 110 of the tubular braided implantable endovascular embolization device connects the distal interference member 145a to a distal end of the pushing member 120 (e.g., delivery wire or tube). The tether 145b preferably has an outer diameter sized to provide sufficient radial clearance space when threaded through the inner channel of the non-inverted proximal section 110 allowing unrestricted 360 degrees free rotation of the tubular braided implantable endovascular embolization device 100 relative to the pushing member 120 (e.g., delivery wire or tube). In addition, during detachment the reduced outer diameter of the tether 145b relative to that of the pushing member 120 (e.g., delivery wire or tube) hastens the time for detaching/severing (e.g., thermal heating, mechanical, or otherwise) to release the pushing member 120 (e.g., delivery wire or tube) from the embolization device 100 remaining implanted at the target site (e.g., in the aneurysm, blood vessel, etc.). The distal interference member 145a and severed portion of the tether 145b attached thereto remains in place within the detached implanted embolization device when the pushing member 120 (e.g., delivery wire or tube) and microcatheter 125, either independently in series one after the other or simultaneously together, are withdrawn from the body. Other configurations for establishing an unrestricted 360 degrees freely rotatable connection of the tubular braided implantable endovascular embolization device relative to the pushing member are possible and within the scope of the present disclosure.

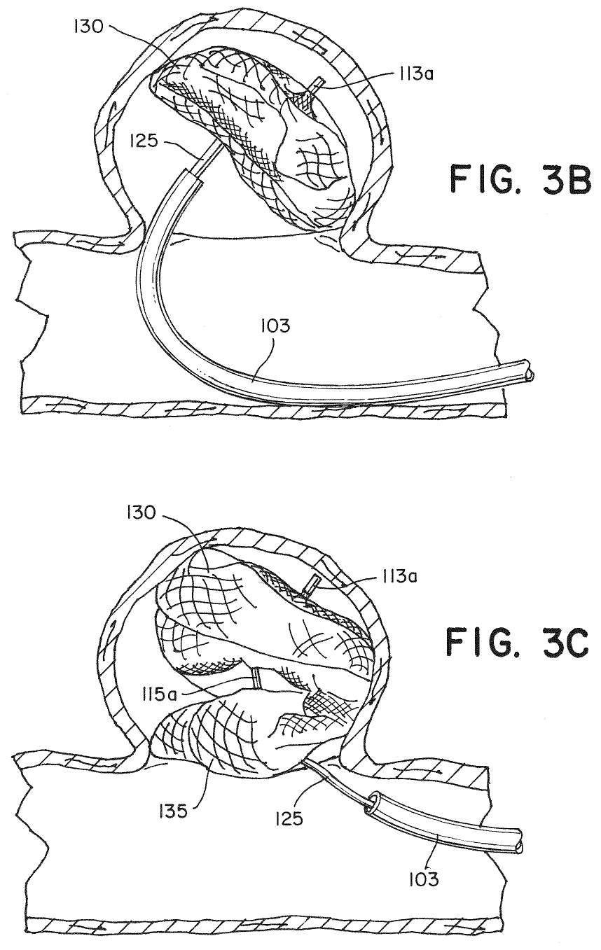

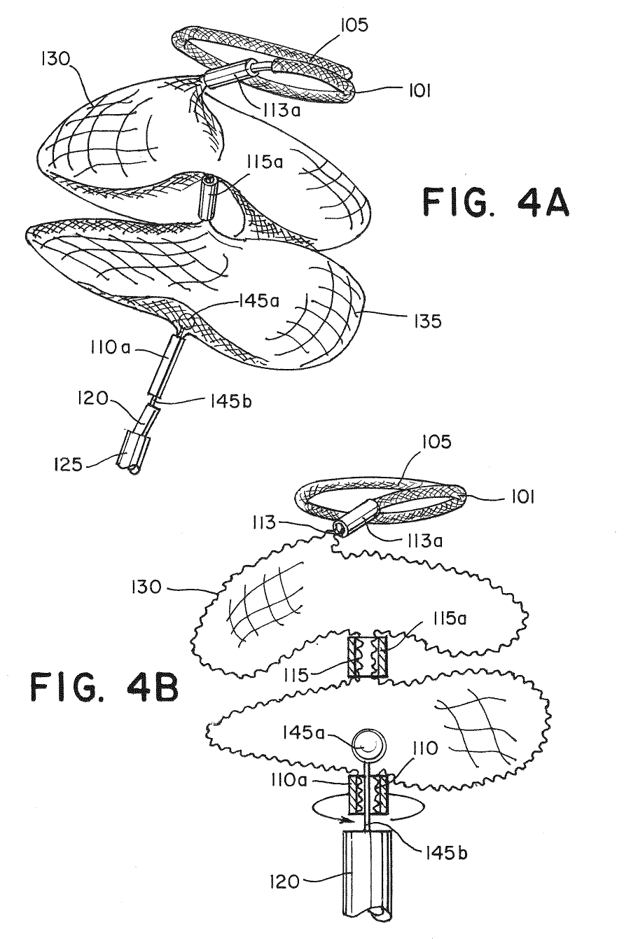

FIGS. 2A-2C & 3A-3C depict sequential stages during delivery through the microcatheter 125 and deployment of the tubular braided implantable endovascular embolization device having a single layer sealing section (as in the example of FIGS. 1A-1C) in treatment of an aneurysm. Specifically, FIGS. 2A-2C depict the sequential stages of deployment of the embolization device over a larger area of vasculature during the treatment of an aneurysm, whereas FIGS. 3A-3C depict enlarged views focusing on the target aneurysm itself illustrating the sequential stages of deployment of the same embolization device. It is noted that the tubular braided implantable endovascular embolization device, regardless of the number of nested layers comprising the multi-layer sealing section (e.g., single layer sealing section (FIGS. 1A-1C), double layer sealing section (FIG. 1D), etc.) undergoes the same sequential stages of delivery and deployment depicted in FIGS. 2A-2C & 3A-3C. By way of illustrative example, FIGS. 2A-2C & 3A-3C depict the delivery and deployment of the tubular braided implantable endovascular embolization device in the treatment of an aneurysm. These same sequential stages of delivery and deployment of the tubular implantable endovascular embolization device in FIGS. 2A-2C & 3A-3C are also applicable in the treatment of restricting or impeding of blood flow in a vessel (e.g., a vessel in the brain or peripheral vasculature). Initially, a guide wire and guide catheter 103 are navigated, either independently in series or simultaneously together, through the vasculature. The guidewire is then withdrawn in a proximal direction while the guide catheter 103 remains in place within the vasculature. Alternatively, the guide catheter 103 may be eliminated altogether wherein the guide wire and microcatheter 125 are navigated, either independently in series or simultaneously together, through the vasculature followed thereafter by subsequent withdraw of the guidewire. In the illustrative example in FIGS. 2A-2C & 3A-3C, the microcatheter 125 is advanced through the guide catheter 103 and out from its distal most end/tip at a proximal face or side of the aneurysm. Next, the tubular braided implantable endovascular embolization device while radially constrained within the lumen of the microcatheter 125 is advanced (i.e., pushed) in a distal direction using the pushing member 120 (e.g., delivery wire or tube). While in the radially constricted state, the stabilizing section 130 and sealing section 135 are reduced in outer diameter smaller than or equal to the inner diameter of the lumen of the microcatheter 125. Each of the non-inverted distal section 113, the intermediate section 115 and the non-inverted proximal section 110 has a narrow outer diameter that is: (i) smaller relative to that of the natural, default, original state radially expanded of maximum outer diameter of either the stabilizing section 130 or the sealing section 135; and (ii) less than or equal to the inner diameter of the lumen of the microcatheter through which the embolization device is deliverable to the target site. Thus, preferably no further radial constriction (i.e., no further reduction in outer diameter) is imposed on the non-inverted distal section 113, the intermediate section 115 or the non-inverted proximal section 110 of the embolization device when introduced and advanced through the lumen of the microcatheter 125 and hence undergo no radial expansion upon exiting from the distal end/tip thereof. The first to emerge from the distal end/tip of the microcatheter 125 is the non-inverted distal section 113 followed by the stabilizing section 130 (FIGS. 2A & 3A). Upon exiting from the distal end/tip of the microcatheter 125 the stabilizing section 130 automatically self-expands radially to an implanted shape and size dependent on the anatomy (e.g., shape and size) of the aneurysm in which it is implanted (depicting partial radial expansion as the stabilizing section 130 partially emerges from the microcatheter 125). FIGS. 2B & 3B depict the stabilizing section 130 fully exited from the distal end/tip of the microcatheter 125 with regions of the surface of the stabilizing section 130 being in direct physical contact imposing a force against the wall of the sac/dome of the aneurysm. Additional advancement (i.e., pushing in the distal direction) of the delivery wire 120 assisted by the heightened stiffness of the intermediate section 115 advances the stabilizing section 130 further distally in the sac/dome of the aneurysm maximizing direct physical surface contact (i.e., coverage) of the stabilizing section 130 with the wall of the sac/dome of the aneurysm (FIG. 2B). Maximized coverage of the aneurysm wall of the sac/dome provided by the stabilizing section 130 is particularly advantageous if the aneurysm is ruptured. Following the intermediate section 115 that doesn't undergo radial expansion upon exiting from the distal end/tip of the microcatheter 125, the sealing section 135 is next to emerge self-expanding radially to its implanted state dependent on the anatomy (e.g., shape and size) of the aneurysm in which it is implanted while spanning the entrance (i.e., neck) (FIGS. 2C & 3C). Thereafter, the non-inverted proximal section 110 of the implanted embolization device fully exits from the distal end/tip of the microcatheter with the sealing section 135 properly seated at the neck of the aneurysm. While in an implanted state (i.e., deployed or radially self-expanded) the sealing section 135 is repositionable to be properly seated at the neck of the aneurysm to maximize diversion or disruption of blood flow. In this regard, the intermediate section 115 (narrower in outer diameter and stiffer (i.e., more rigid) relative to that of the stabilizing section 130 while in a radially expanded implanted state) acts like a hinge allowing independent and angular offset positioning of the sealing section 135 relative to that of the stabilizing section 130 anchored in place. While visible under imaging (e.g., fluoroscopic imagery), proper positioning of the sealing section 135 at the neck of the aneurysm is realized by the physician or interventionalist manipulating (e.g., partially retracting or pulling in a proximal direction vs. partially advancing or pushing in a distal direction) the pushing member 120 (e.g., delivery wire or tube). When properly seated at the neck of the aneurysm, as illustrated in FIGS. 2C & 3C, preferably only a minimal portion (e.g., ≤approximately 5%) of the sealing section 135 protrudes or extends into the adjacent parent vessel, i.e., substantially all (e.g., ≥approximately 95%) of the sealing section 135 is disposed within the aneurysm (i.e., neck or sac/dome). Lastly, detachment takes place by detaching or severing (e.g., via electrolytic and/or mechanical detachment) the tether 145b thereby releasing the delivery wire 120. Following release, the pushing member 120 (e.g., delivery wire or tube) and microcatheter 125, either independently in series one after the other or simultaneously together, are withdrawn in a proximal direction from the body. Detached from the pushing member 120 (e.g., delivery wire or tube), the implantable endovascular embolization device (together with the distal interference member 145a and severed portion of the tether 145b) remains implanted in the aneurysm. Alternative delivery systems and detachment mechanisms are contemplated and within the scope of the present invention.

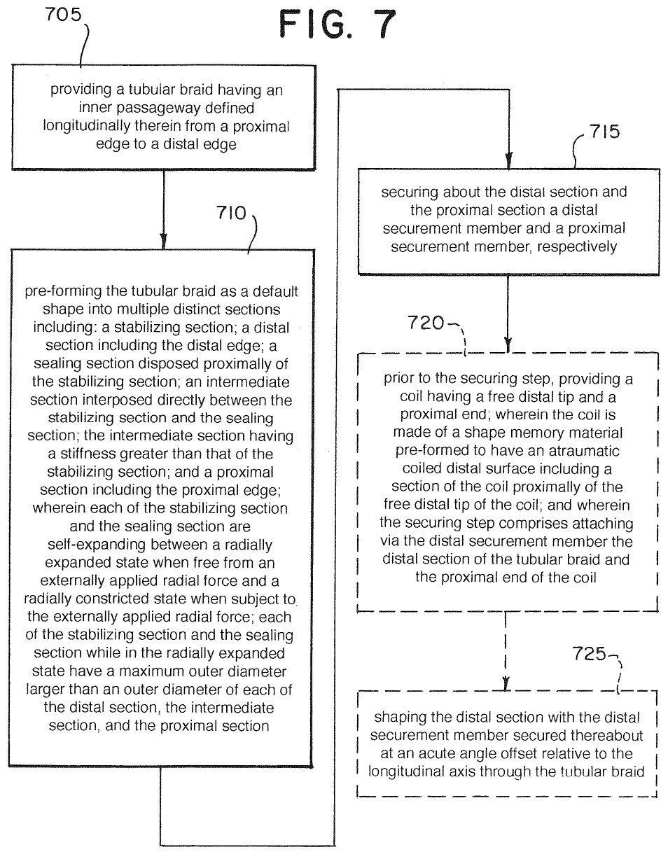

In the exemplary multi-sectional tubular implantable endovascular embolization device of FIGS. 1A-1C described above both the distal section 113 and the proximal section 110 are non-inverted (i.e., neither the distal edge nor the proximal edge is inverted inwardly in the internal passageway defined in the axial/longitudinal direction of the tubular braid). Accordingly, the distal edge of the non-inverted distal section 113 projects distally relative to the stabilizing section 130, while the proximal edge of the non-inverted proximal section 110 projects proximally relative to the sealing section 135. The same features (e.g., stabilizing section 130, intermediate section 115 and sealing section 135) as described above are present in an alternative inverted configuration of the tubular braided implantable endovascular embolization device shown in FIGS. 8A-8E with the only difference being that each of the distal and proximal sections are inverted inwardly into the internal passageway defined in the axial/longitudinal direction of the tubular braid. Specifically, the inverted distal section 113′ is fully socked inwardly through the inner passage 140 of the tubular braid until fully disposed within the interior space of the stabilizing section 130, while the inverted proximal section 110′ is fully socked inwardly through the inner passage 140 of the tubular braid until fully disposed within the interior space of the sealing section 135. A distal securement member 113a (e.g., distal marker band) is physically secured about the inverted distal section 113′, while a proximal securement member 110a (e.g., proximal marker band) is physically secured about the inverted proximal section 110′ together with the distal end of the pushing member 120 (e.g., delivery wire or tube). Inverting the distal section 113′ advantageously creates an atraumatic distal surface that when deployed at the target site (e.g., aneurysm, blood vessel, etc.) minimizes risk of damage to the blood vessel while also maximizing the contact surface area of the stabilizing section 130 with the inner wall of the aneurysm. While inverting the proximal section 110′ provides the benefit of ensuring that the proximal securement member 110a (e.g., proximal marker band) does not extend into the healthy parent vessel. The non-limiting examples depicted in FIGS. 8A-8E represent different variations in size among the stabilizing and sealing section 130, 135, respectively, in either the x-direction or the y-direction (as represented by the coordinate system legend, wherein the y-direction extends longitudinally axially through the implant and the x-direction is perpendicular thereto). In the illustrative examples in FIGS. 8A-8C the stabilizing and sealing sections 130, 135, respectively, are all substantially equal in the x-direction, differing from one another only in the y-direction (e.g., FIG. 8A—the stabilizing and sealing sections 130, 135, respectively, are substantially equal in y-direction; FIG. 8B—the stabilizing section 130<the sealing section 135 in the y-direction; FIG. 8C—the stabilizing section 130>the sealing section 135 in the y-direction). While in the illustrative examples in FIGS. 8D-8E the stabilizing and sealing sections 130, 135, respectively, are all substantially equal in the y-direction, differing from one another only the x-direction (e.g., FIG. 8D—the stabilizing section 130<the sealing section 135 in the x-direction; FIG. 8E—the stabilizing section 130>the sealing section 135 in the x-direction). These illustrative examples are not exhaustive, other variations are contemplated in which the stabilizing and sealing sections 130, 135, respectively, are unequal in size from each other in both the x-direction and the y-direction. Moreover, the implant in FIGS. 8A-8E includes both an inverted distal section 113′ and an inverted proximal section 110′, however, only one or the other (i.e., inverted distal section 113′ or inverted proximal section 110′) is also within the scope of the present disclosure. The implant having inverted distal and/or proximal sections optionally may also have a sealing section 135 with multiple nested layers and preferably is unrestricted 360 degrees freely rotatable relative to the pushing member 120 (e.g., delivery wire or tube). Any of the implants having inverted distal and/or proximal sections (FIGS. 8A-8E) undergo similar sequential stages of deployment of the respective stabilizing 130, intermediate 115 and sealing sections 135 as they emerge from the distal end of the microcatheter 125 in a similar manner represented in FIGS. 2A-2C & 3A-3C for the implant having non-inverted distal and proximal sections. The only difference being that upon exiting from the microcatheter 125, the distal most end of the embolization device includes both the stabilizing section 130 together with the inverted distal section 113 disposed in the interior space thereof, while the proximal most end of the embolization device includes both the sealing section 135 together with the inverted proximal section 110 disposed in the interior space thereof.

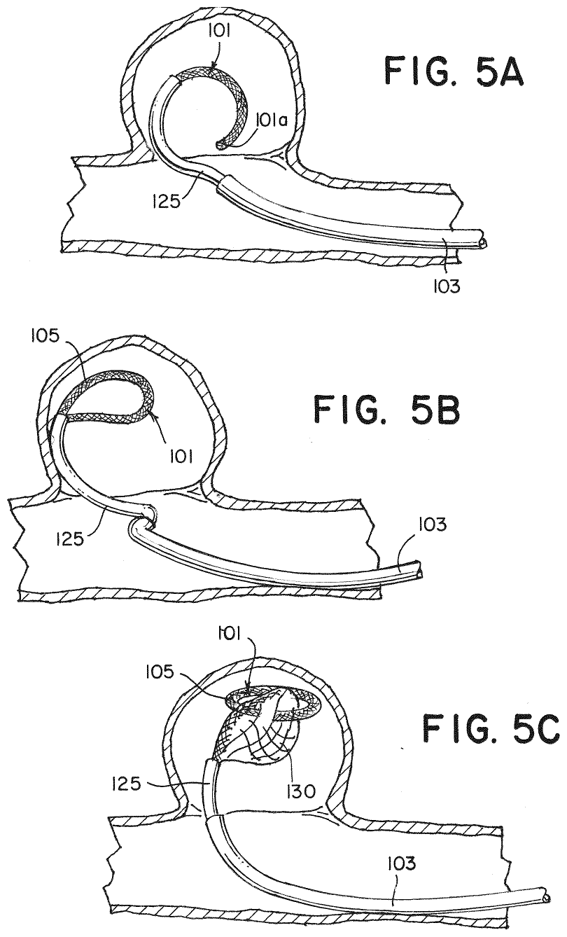

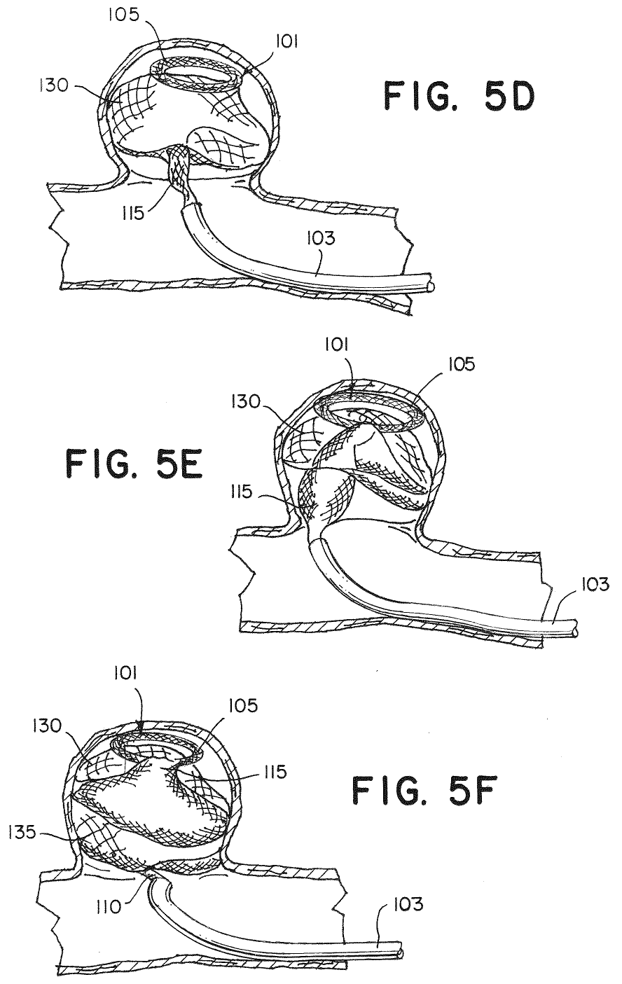

Still another exemplary tubular braided implantable endovascular embolization device is shown in FIG. 4A. The implant in FIG. 4A is identical to that in FIG. 1A, as described above, the only difference being the addition of a single coil 101 projecting distally from the distal end of the non-inverted distal section 113 physically secured via the distal marker band 113a or other conventional securement mechanism (e.g., suture, adhesive, weld, etc.). Any example in accordance with the present disclosure may incorporate the single coil 101 providing several beneficial aspects to the device. That portion of the single coil 101 projecting distally from the stabilizing section 130 beneficially provides added/extra material and contact surface area for potential clot formation in the aneurysm (especially at the dome of the dome of the aneurysm), while the inverted portion relieves the pressure imposed by the coil on the wall of the aneurysm. Coil 101 is preferably made of a shape memory alloy (e.g., Nitinol—alloy of nickel and titanium) having a pre-formed (e.g., heat set) natural, original, default, or coiled configuration with a maximum outer diameter and minimum length in the axial/longitudinal direction. While manipulating (e.g., pushing in the distal direction and/or pulling in the proximal direction) the deployed implant so that the sealing section 135 is properly seated at the neck of the aneurysm, in its pre-formed natural, original, default or coiled configuration the coil 101 acts as a bumper minimizing risk of damage to the vessel wall (e.g., wall of the aneurysm cavity, blood vessel, etc.). In the pre-formed natural, original, default, or coiled configuration the distal coil 101 forms an atraumatic distal coiled loop surface 105 maximizing contact surface area with and thus distributing (i.e., spreading out) the force imposed on the inner wall of the aneurysm cavity. The atraumatic distal coiled loop surface 105 comprises a section of the coil 101 proximally of the distal tip/end 101a but optionally may include the distal tip/end 101a itself. In the example illustrated in FIGS. 4A & 4B, the atraumatic distal coiled loop surface 105 (e.g., resembling that of a “halo”) includes at least a section of which is planar or flat (i.e., substantially parallel to a radial cross-sectional axis through the maximum outer diameter of the stabilizing section 130 while in the radially expanded (i.e., deployed) state. Alternatively, the atraumatic distal coiled loop surface 105 may be curved, domed, or hemispherical (i.e., free from any planar or flat section). Regardless of the contour or profile, preferably no portion of the coil 101 or any other component of the tubular braided implantable endovascular embolization device 100 extends distally beyond the atraumatic distal coiled loop surface 105. During delivery through the lumen of the microcatheter 125 to the target site the coil 101 is in the deformed (i.e., uncoiled or outstretched in a longitudinal/axial direction) state having a reduced outer diameter and expanded in length in an axial/longitudinal direction relative to that while in the preformed natural, original, or coiled configuration. As the distal end/tip 101a of the coil 101 first to exit from the distal tip/end of the microcatheter 125 the coil 101 automatically reverts or returns to its preformed natural, original, default, or coiled configuration. Once the implant is deployed at the target site (e.g., in the cavity of the aneurysm) the atraumatic distal coiled loop surface 105 acting as a bumper minimizes risk of damage to the cavity wall while manipulating (e.g., pushing in the distal direction and/or pulling in the proximal direction) the pushing member 120 (e.g., delivery wire or tube) while properly seating the sealing section 135 at the neck of the aneurysm. The implant of FIGS. 4A-4B having the distal coil 101 optionally may also have a sealing section 135 comprising multiple nested layers and preferably is unrestricted 360 degrees freely rotatable relative to the delivery wire 120.

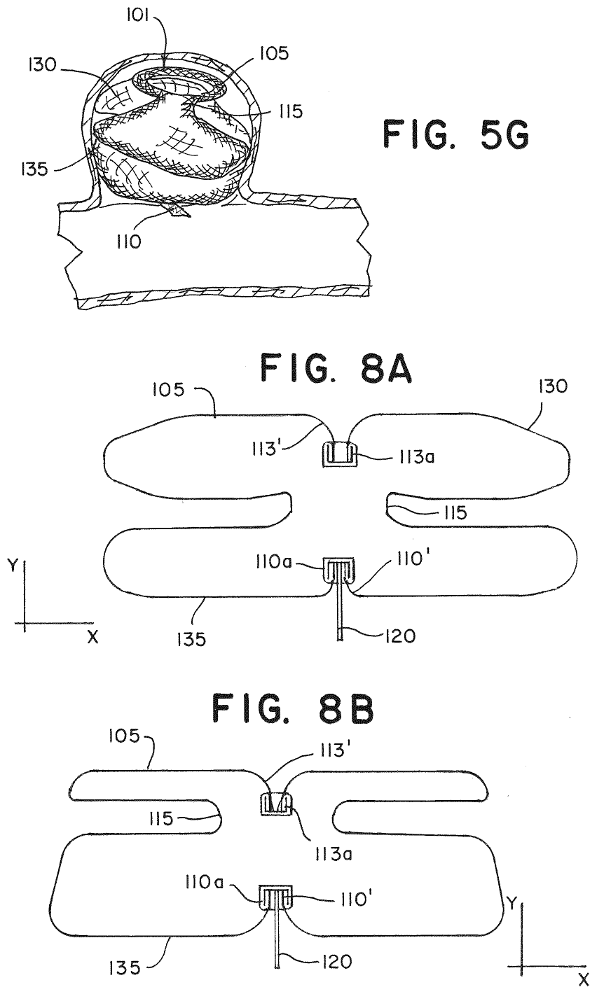

FIGS. 5A-5G depict enlarged views of the sequential stages of deployment of the multi section tubular braided implant having a distal coil 101 as shown in FIGS. 4A-4B during the treatment of an aneurysm. By way of illustrative example, FIGS. 5A-5G depict the delivery and deployment of the tubular braided implantable endovascular embolization device in the treatment of an aneurysm. These same sequential stages of delivery and deployment of the tubular implantable endovascular embolization device in FIGS. 4A-4B are also applicable in the treatment of restricting or impeding of blood flow in a vessel (e.g., a vessel in the brain or peripheral vasculature). Initially, a guide wire and guide catheter 103 are navigated, either independently in series or simultaneously together, through the vasculature. The guidewire is then withdrawn in a proximal direction while the guide catheter 103 remains in place within the vasculature. Alternatively, the guide catheter 103 may be eliminated altogether wherein the guide wire and microcatheter 125 are navigated, either independently in series or simultaneously together, through the vasculature followed thereafter by subsequent withdraw of the guidewire. In the illustrative example in FIGS. 4A-4B, the microcatheter 125 is advanced through the guide catheter 103 and out from its distal most end/tip at a proximal face or side of the aneurysm. Next, the implant is advanced (i.e., pushed) in a distal direction via the pushing member 120 (e.g., delivery wire or tube) through the microcatheter 125 simultaneously while the distal coil 101 is in a longitudinally/axially extended state and both the stabilizing and the sealing sections 130, 135, respectively, of the tubular braided implantable endovascular embolization device 100 are radially constrained. Each of the non-inverted distal section 110, the intermediate section 115 and the non-inverted proximal section 110 has a narrow outer diameter that is: (i) smaller relative to that of the natural, default, original state radially expanded of maximum outer diameter of either the stabilizing section 130 or the sealing section 135; and (ii) less than or equal to the inner diameter of the lumen of the microcatheter 125 through which the embolization device is deliverable to the target site. Thus, preferably no further radial constriction (i.e., no further reduction in outer diameter) is imposed on the non-inverted distal section 113, the intermediate section 115 or the non-inverted proximal section 110 of the embolization device when introduced and subsequently advanced through the lumen of the microcatheter 125 and therefore experience no radial expansion upon exiting from the distal end/tip thereof. Referring to FIG. 5A, when being advanced through the microcatheter 125 while the coil 101 is in an extended longitudinal/axial state of reduced outer diameter the distal most portion of the implant first to exit is the distal tip/end 101a of the coil (the atraumatic distal coiled surface 105 representing the distal most portion only in the pre-formed original, natural, default state). With continued advancement in the distal direction the distal coil 101 as it emerges from the microcatheter 125 automatically reverts or returns to its pre-formed original, natural, or default shape including the atraumatic distal coiled surface 105 that when fully deployed represents the distal most surface of the implant in direct physically contact with the inner wall of the aneurysm cavity (e.g., substantially opposite that of the neck)(FIG. 5B). Despite such physical engagement, risk of injury to the thin wall of the aneurysm cavity is minimized by designing the distal coiled surface 105 representing the distal most portion of the deployed implant to be atraumatic while also distributing the imposed force over an expanded contact surface including a section of the coil 101 proximally of its distal tip/end 101a. Next to emerge from the distal end/tip of the microcatheter 125 is the non-inverted distal section 113 followed by the stabilizing section 130 (FIG. 5C). Upon exiting from the distal end/tip of the microcatheter 125 the stabilizing section 130 automatically self-expands radially to an implanted shape and size dependent on the anatomy (e.g., shape and size) of the aneurysm in which it is implanted (depicting partial radial expansion as the stabilizing section 130 partially emerges from the microcatheter 125). FIG. 5D shows the stabilizing section 130 fully exited from the distal end/tip of the microcatheter 125 with regions of the surface of the stabilizing section 130 being in direct physical contact imposing a force against the wall of the sac/dome of the aneurysm stabilized or anchored in place. Following the intermediate section 115 that preferably doesn't undergo radial expansion upon exiting from the distal end/tip of the microcatheter 125 (FIG. 5E), the sealing section 135 is next to emerge self-expanding radially to its implanted state dependent on the anatomy (e.g., shape and size) of the aneurysm in which it is implanted while spanning the entrance (i.e., neck) (FIG. 5F). Thereafter, the non-inverted proximal section 110 of the implanted embolization device fully exits from the distal end/tip of the microcatheter with the sealing section 135 properly seated at the neck of the aneurysm. While in an implanted state (i.e., deployed or radially self-expanded) the sealing section 135 is repositionable to be properly seated at the neck of the aneurysm to maximize diversion or disruption of blood flow. In this regard, the intermediate section 115 (narrower in outer diameter and stiffer (i.e., more rigid) relative to that of the stabilizing section 130 while in a radially expanded implanted state) acts like a hinge allowing independent and angular offset positioning of the sealing section 135 relative to that of the stabilizing section 130 anchored in place. While visible under imaging (e.g., fluoroscopic imagery), proper positioning of the sealing section 135 at the neck of the aneurysm is realized by the physician or interventionalist manipulating (e.g., partially retracting or pulling in a proximal direction vs. partially advancing or pushing in a distal direction) the pushing member 120 (e.g., delivery wire or tube). It is during such repositioning at the target site (e.g., in the aneurysm cavity) that the atraumatic distal coiled surface 105 of the distal coil 101 while in its pre-formed state serves as a bumper minimizing injury to the wall of the aneurysm. When properly seated at the neck of the aneurysm preferably only a minimal portion (e.g., ≤approximately 5%) of the sealing section 135 protrudes or extends into the adjacent parent vessel, i.e., substantially all (e.g., ≥approximately 95%) of the sealing section 135 is disposed within the aneurysm (i.e., neck or sac/dome). Lastly, detachment (e.g., electrolytic and/or mechanical) takes place detaching (e.g., severing or releasing) the tether 145b from the pushing member 120 (e.g., delivery wire or tube)(FIG. 5G). Following release, the pushing member 120 and microcatheter 125, either independently in series one after the other or simultaneously together, are withdrawn in a proximal direction from the body. Detached from the pushing member 120 (e.g., delivery wire or tube), the implantable endovascular embolization device (together with the distal interference member 145a and severed portion of the tether 145b) remains implanted at the target site (e.g., in the aneurysm). Alternative delivery systems and detachment mechanisms are contemplated and within the scope of the present invention.

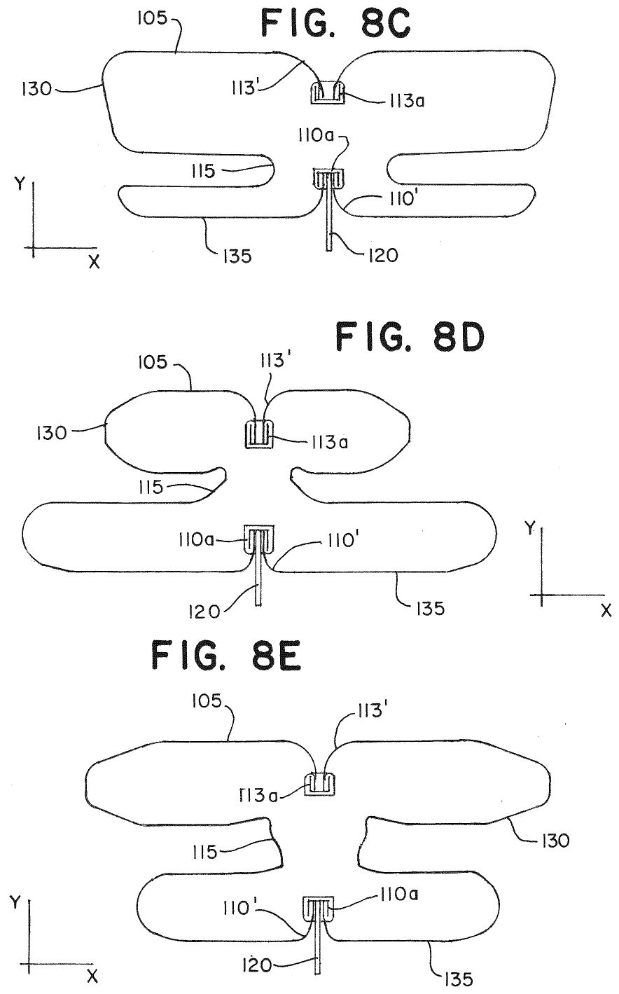

Operation of the tubular braided implantable endovascular embolization device in accordance with the present disclosure is set forth in the flow chart of FIG. 6. Initially in step 605 the microcatheter 125 is navigated through a vasculature to the target site. This step may be accomplished in several different ways depending on the catheter delivery system used. In one example, a guide wire and microcatheter 125 are navigated, independently in series or simultaneously together, through the vasculature to the target site (e.g., on a proximal side of the aneurysm or blood vessel (e.g., in the brain or periphery vessel)) followed by subsequent removal of the guide wire while the microcatheter 125 remains in place within the vasculature. Alternatively, a guide catheter 103 and guide wire, either independently in series one after the other or simultaneously together, are navigated through the vasculature with subsequent withdraw of the guide wire followed the microcatheter 125 advanced through until emerging from the distal end/tip of the guide catheter 103. While in the radially constricted state, in step 610 the tubular braided implantable endovascular aneurysm embolization device is advanced (e.g., pushed) in a distal direction through the microcatheter 125 using the pushing member 120 (e.g., delivery wire or tube). In all configurations (e.g., without the distal coil 101 (regardless of whether the distal and/or proximal ends are non-inverted or inverted) or with the distal coil 101) while in a radially constricted state the stabilizing and sealing sections 130, 135, respectively, are advanced through the lumen of the microcatheter 125. Optionally, in the specific configuration of the implant including the distal coil 101, during delivery through the lumen of the microcatheter 125 the distal coil 101 is radially constrained (i.e., the coil is extended in a longitudinal/axial direction having a reduced outer diameter)(optional step 625 depicted within the dashed-line box). In step 615, upon exiting from a distal end/tip of the microcatheter 125 the tubular braided implantable endovascular embolization device 100 is deployed at the target site (e.g., in the aneurysm or blood vessel). Upon emerging or exiting from the distal end/tip of the microcatheter 125 the stabilizing section 130 automatically self-expands radially to an implanted state dependent on the anatomy of the target site (e.g., aneurysm or blood vessel). Optionally, if the implant includes the distal coil 101, then upon emerging or exiting from the distal end/tip of the microcatheter 125 the distal coil 101 automatically reverts or returns to its pre-formed coiled state radially expanded in outer diameter including the atraumatic distal coiled surface 105 (optional step 625 depicted within the dashed-line box). In an implanted state, the stabilizing section 130 is in direct physical contact imposing a force against the vessel wall (e.g., sac/dome wall of the aneurysm) stabilizing, anchoring, or maintaining its position therein. Continued pushing of the implant 100 in the distal direction advances the intermediate section 115 from the distal end/tip of the microcatheter 125 preferably without undergoing radial expansion (i.e., substantially unchanging in outer diameter). The intermediate section 115 provides column strength between while also acting as a hinged connection point allowing independent positioning between the stabilizing section 130 and the sealing section 135. Next to emerge from the distal end/tip of the delivery device (e.g., microcatheter 125), the sealing section 135 radially self-expands spanning the neck of the aneurysm diverting, disrupting, or impeding blood flow therethrough to the target site (e.g., aneurysm or blood vessel). In the treatment of an aneurysm, the sealing section 135 provides sufficient, but not complete (i.e., total or full), diversion or disruption of blood flow therethrough to optimize healing at the neck of the aneurysm. The stabilizing section 130 while anchored in place together with the intermediate section 115 (enhanced in stiffness) pushes the sealing section 135 towards thereby stabilizing at the neck and as a result minimizing risk of distal migration (e.g., into the aneurysm) over time (i.e., future recanalization). While implanted and subject to visible imagery (e.g., fluoroscopic imagery), if necessary, the physician or interventionalist may physically manipulate (e.g., partially retracting/pulling in a proximal direction and/or advancing/pushing in a distal direction) the pushing member 120 (e.g., delivery wire or tube) to properly seat the sealing section 135 at the target site (e.g., neck of the aneurysm or blood vessel) thereby maximizing diversion or occlusion of blood flow. In the exemplary implant with the optional distal coil 101, the atraumatic distal coiled surface 105 serves as a bumper to minimize risk of damage of the thin wall of the aneurysm cavity. Now with the implant properly positioned at the target site, in step 620 the tether 145b is detached (e.g., physically released or electrolytically severed) remaining in place at the target site together with the tubular braided implantable endovascular embolization device while the delivery wire 120 and microcatheter 125 either independently or simultaneously, are withdrawn in a proximal direction from the body.

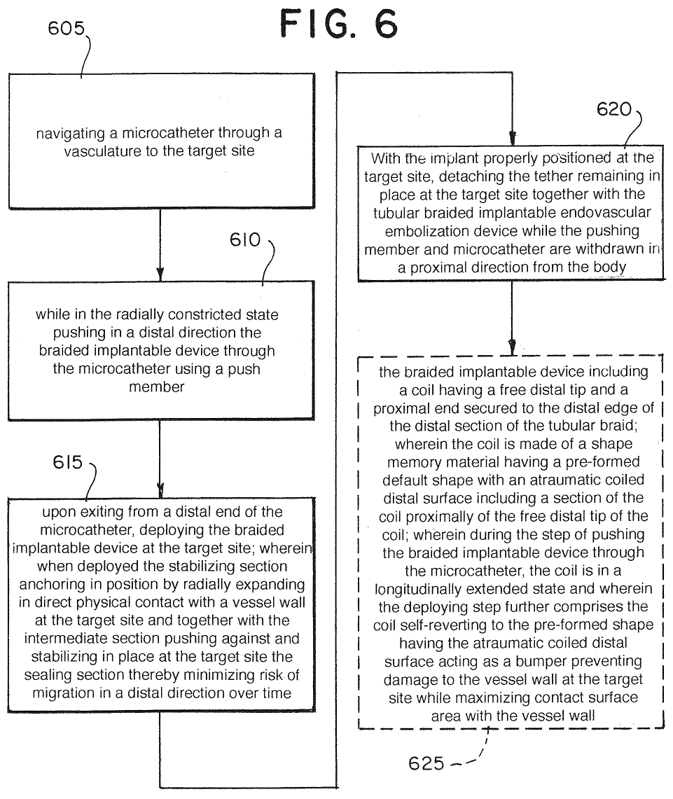

The exemplary flow chart in FIG. 7 depicts the steps in the manufacture of the multi-section tubular braided implantable endovascular embolization device of FIG. 1A (having non-inverted distal and proximal sections 113, 110, respectively, and a single layer sealing section 135) or 8A-8E (having inverted distal and proximal sections 113′, 110′, respectively). Initially, a cylindrical tubular braided structure (preferably a right cylinder) is provided in step 705 comprising a plurality of wires (each wire preferably having an outer diameter of ≤approximately 0.001″) woven into a desired pattern (e.g., full diamond pattern). The formed cylindrical tubular braid forms an inner passageway 140 extending in the longitudinal/axial direction between edges at opposing distal and proximal ends of the cylindrical tubular braided structure. Next in step 710, the cylindrical tubular braided structure is pre-formed (e.g., heat set) to include the following multiple distinct sections: distal section (non-inverted 113 or inverted 113′); stabilizing section 130; intermediate section 115; sealing section 135; and proximal section (non-inverted 110 or inverted 110'). If the sealing section 135 includes more than one braided layer 135a, 135b then optionally one or more supplemental structural obstructing layer(s) 135b (e.g., supplemental braided layer(s), supplemental coil(s), or supplemental suture(s)) may be nested within the to be formed sealing section 135a of the cylindrical tubular braided structure in advance of pre-forming step 710. Each of the stabilizing and sealing sections 130, 135, respectively, are pre-formed to be radially self-expanding (i.e., radially collapsible or constricted during delivery to the target site through the microcatheter 125, and upon exiting from the distal end/tip of the microcatheter 125 are radially self-expandable to an implanted size and shape conforming to the anatomy (e.g., size and shape) of the target site (e.g., sac/dome of the aneurysm, blood vessel, etc.). The stabilizing and sealing sections 130, 135, respectively, may, but need not necessarily, have the same maximum outer diameter while in the pre-formed natural, default, or original state (e.g., radially expanded state) free from any externally radially constricting force. Distal section (non-inverted 113 or inverted 113′), intermediate section 115 and proximal section (non-inverted 110 or inverted 110′) each have a narrow outer diameter that is: (i) smaller relative to that of the pre-formed natural, default, original state radially expanded of maximum outer diameter of either the stabilizing section 130 or the sealing section 135; and (ii) less than or equal to the inner diameter of the lumen of the microcatheter 125 through which the embolization device is deliverable to the target site. Regardless of the state (e.g., radially expanded or radially contracted) of any other section of the embolization device at any given time, the outer diameter preferably is maintained substantially unchanged for each of the distal section (non-inverted 113 or inverted 113′) the intermediate section 115 and the proximal section (non-inverted 110 inverted 110′). Then in step 715 the distal securement member 113a (e.g., distal marker band) is secured (e.g., physically crimped or deformed) about the distal edge of the cylindrical tubular braided structure, while the proximal securement member 110a (e.g., proximal maker band) is secured (e.g., physically crimped or deformed) about the proximal edge of the cylindrical tubular braided structure along with the tether 145b which, in turn, is attached (permanently or releasably) to the pushing member 120 (e.g., delivery wire or tube). The tubular braided implantable endovascular embolization device 100 and the pushing member 120 is preferably secured to one another via the unrestricted 360 degrees freely rotatably connection as in the example of FIGS. 1A-1C, but otherwise may be fixedly (i.e., non-rotatably) connected as in example of FIGS. 8A-8E). The type of delivery system (e.g., pushing member) and connection of the implant therewith may be modified, as desired, but an unrestricted 360 degrees freely rotatable connection of the implant relative to the delivery system (e.g., pushing member 120) is preferred. In the configuration in which the distal section 113′ and/or proximal section 110′ of the cylindrical tubular braided structure are inverted inwardly within the interior space of the stabilizing and sealing sections, respectively, inversion would occur in advance of securement of the securement member (e.g., marker band) in step 715. Optionally, if the tubular braided implantable endovascular embolization device includes a coil (FIGS. 4A-4B), prior to securing step 715, in steps 720 & 725 the distal coil 101 is shaped (heat set) and then secured via the distal securement member 113a (e.g., distal marker band) to the distal section 113 of the braided structure followed by subsequent forming of the atraumatic distal surface 105 (e.g., resembling a “halo”) and offset formation of the distal section 113 together with the distal securement member 113a secured thereabout relative to the longitudinal axis through the tubular braid. By way of example, such forming step may be realized by placing the coil 101 in a spherical shape mold.

Aspects of the present disclosure are also provided by the following numbered Clauses:
Clause 1: An endovascular embolization system comprising: a braided implantable device (100) comprising: a tubular braid having an inner passageway (140) defined longitudinally therein from a proximal edge to a distal edge; wherein the tubular braid is pre-formed as a default shape into multiple distinct sections including: a stabilizing section (130); a distal section (113, 113′) including the distal edge; a sealing section (135) disposed proximally of the stabilizing section (130); an intermediate section (115) interposed directly between the stabilizing section (130) and the sealing section (135); the intermediate section (115) having a stiffness greater than that of the stabilizing section (130); and a proximal section (110, 110′) including the proximal edge; wherein each of the stabilizing section (130) and the sealing section (135) are self-expanding between a radially expanded state when free from an externally applied radial force and a radially constricted state when subject to the externally applied radial force; each of the stabilizing section (130) and the sealing section (135) while in the radially expanded state have a maximum outer diameter larger than an outer diameter of each of the distal section (113, 113′), the intermediate section (115), and the proximal section (110, 110′).
Clause 2: The system of Clause 1, wherein the braided implantable device further comprises a coil (101) having a free distal tip (101a) and a proximal end secured to the distal section (113, 113′) of the tubular braid; wherein the coil (101) is made of a shape memory material pre-formed to have an atraumatic coiled distal surface (105) including a section of the coil (101) proximally of the free distal tip (101a) of the coil (101).
Clause 3: The system of Clause 2, wherein the distal edge of the distal section (113, 113′) of the tubular braid and the proximal end of the coil (101) are secured via a distal securement member (110a); and in the pre-formed default shape the distal section (113, 113′) of the tubular braid is offset at an acute angle relative to a longitudinal axis through the tubular braid.
Clause 4: The system of any of Clauses 1 through 3, further comprising: a distal securement member (113a) fixedly secured about the distal section (113, 113′) of the tubular braid; and
   a proximal securement member (110a) fixedly secured about the proximal section (110, 110′) of the tubular braid.
Clause 5: The system of any of Clauses 1 through 4, wherein the distal section (113, 113′) including the distal edge is disposed distally or interiorly of the stabilizing section (130); and the proximal section (110, 110′) including the proximal edge is disposed proximally or interiorly of the sealing section (135).
Clause 6: The system of any of Clauses 2 through 5, wherein at least a portion of the atraumatic coiled distal surface (105) is substantially perpendicular to a longitudinal axis through the tubular braid.
Clause 7: A method for disrupting blood flow to a target site using an endovascular embolization system that includes a braided implantable device (100) comprising: a tubular braid having an inner passageway (140) defined longitudinally therein from a proximal edge to a distal edge; wherein the tubular braid is pre-formed as a default shape into multiple distinct sections including: a stabilizing section (130); a distal section (113, 113′) including the distal edge; a sealing section (135) disposed proximally of the stabilizing section (130); an intermediate section (115) interposed directly between the stabilizing section (130) and the sealing section (135); the intermediate section (115) having a stiffness greater than that of the stabilizing section (130); and a proximal section (110, 110′) including the proximal edge; wherein each of the stabilizing section (130) and the sealing section (135) are self-expanding between a radially expanded state when free from an externally applied radial force and a radially constricted state when subject to the externally applied radial force; each of the stabilizing section (130) and the sealing section (135) while in the radially expanded state have a maximum outer diameter larger than an outer diameter of each of the distal section (113, 113′), the intermediate section (115), and the proximal section (110, 110′); the method comprising the steps of: navigating a microcatheter (125) through a vasculature to the target site; while in the radially constricted state, pushing in a distal direction the braided implantable device (100) through the microcatheter (125) using a pushing member (120); and upon exiting from a distal end of the microcatheter (125), deploying the braided implantable device (100) at the target site; wherein when deployed the stabilizing section (130) anchoring in position by radially expanding in direct physical contact with a vessel wall at the target site and together with the intermediate section (115) pushing against and stabilizing in place at the target site the sealing section (135) thereby minimizing risk of migration in a distal direction over time.
Clause 8: The method of Clause 7, wherein the braided implantable device further comprises a coil (101) having a free distal tip (101a) and a proximal end secured to the distal edge of the distal section (113, 113′) of the tubular braid; wherein the coil (101) is made of a shape memory material having a pre-formed default shape with an atraumatic coiled distal surface (105) including a section of the coil (101) proximally of the free distal tip (101a) of the coil (101); wherein during the step of pushing the braided implantable device through the microcatheter (125), the coil (101) is in a longitudinally extended state wherein the deploying step further comprises the step of the coil (101) automatically reverting to the pre-formed shape having the atraumatic coiled distal surface (105) acting as a bumper preventing damage to the vessel wall during positioning at the target site while maximizing contact surface area with the vessel wall.
Clause 9: The method of Clause 8, wherein at least a portion of the atraumatic coiled distal surface (105) of the coil (101) is substantially planar.
Clause 10: The method of any of Clauses 8 through 9, wherein the distal edge of the distal section (113, 113′) of the tubular braid and the proximal end of the coil (101) are secured via a distal securement member (110a); and in the pre-formed default shape the distal section (113, 113′) of the tubular braid is offset at an acute angle relative to a longitudinal axis through the tubular braid.
Clause 11: The method of any of Clauses 8 through 10, wherein at least a portion of the atraumatic coiled distal surface (105) is substantially perpendicular to a longitudinal axis through the tubular braid.
Clause 12: The method of any of Clauses 7 through 11, wherein the braided implantable device further comprises: a distal securement member (113a) fixedly secured about the distal section (113, 113′) of the tubular braid; a proximal securement member (110a) fixedly secured about the proximal section (110, 110′) of the tubular braid.
Clause 13: The method of any of Clauses 7 through 12, wherein the distal section (113, 113′) including the distal edge is disposed distally or interiorly of the stabilizing section (130); and the proximal section (110, 110′) including the proximal edge is disposed proximally or interiorly of the sealing section (135).
Clause 14: The method of any of Clauses 7 through 13, wherein during the deploying step neither the distal section (113, 113′) nor the proximal section (110, 110′) expand radially.
Clause 15: A method of manufacture an endovascular embolization system, the method comprising the steps of: providing a tubular braid having an inner passageway (140) defined longitudinally therein from a proximal edge to a distal edge; pre-forming the tubular braid as a default shape into multiple distinct sections including: a stabilizing section (130); a distal section (113, 113′) including the distal edge; a sealing section (135) disposed proximally of the stabilizing section (130); an intermediate section (115) interposed directly between the stabilizing section (130) and the sealing section (135); the intermediate section (115) having a stiffness greater than that of the stabilizing section (130); and a proximal section (110, 110′) including the proximal edge; wherein each of the stabilizing section (130) and the sealing section (135) are self-expanding between a radially expanded state when free from an externally applied radial force and a radially constricted state when subject to the externally applied radial force; each of the stabilizing section (130) and the sealing section (135) while in the radially expanded state have a maximum outer diameter larger than an outer diameter of each of the distal section (113, 113′), the intermediate section (115), and the proximal section (110, 110′).
Clause 16: The method of Clause 15, further comprising securing about the distal section (113, 113′) and the proximal section (110, 110′) a distal securement member (113a) and a proximal securement member (110a), respectively.
Clause 17: The method of any of Clauses 15 through 16, wherein after the pre-forming step and before the securing step further comprising heating to an inverted shape: (i) the distal section (113′) including the distal edge to be disposed interiorly of the stabilizing section (130); and/or (ii) the proximal section (110) including the proximal edge to be disposed interiorly of the sealing section (135).
Clause 18: The method of any of Clauses 15 through 17, wherein the heating step further comprises heating a coil (101) having a free distal tip (101a) and a proximal end; wherein the coil (101) is made of a shape memory material pre-formed to have an atraumatic coiled distal surface (105) including a section of the coil (101) proximally of the free distal tip (101a) of the coil (101); and wherein the securing step comprises attaching via the distal securement member (113a) the distal section (113, 113′) of the tubular braid and the proximal end of the coil (101).
Clause 19: The method of Clause 18, further comprising shaping the distal section (113, 113′) with the distal securement member (113a) secured thereabout at an acute angle offset relative to a longitudinal axis through the tubular braid while simultaneously creating the atraumatic distal surface.
Clause 20: The method of any of Clauses 18 through 19, wherein at least a portion of the atraumatic coiled distal surface (105) is substantially perpendicular to a longitudinal axis through the tubular braid.

The descriptions contained herein are examples and not intended in any way to limit the scope of the present disclosure. As described herein, the present disclosure contemplates many variations and modifications of the tubular braided implantable embolization device having a folded over atraumatic distal end for use in diverting/disrupting blood flow to an aneurysm or impeding/restricting blood flow in a vessel (e.g., in the brain or peripheral vasculature). Modifications and variations apparent to those having skilled in the pertinent art according to the teachings of this disclosure are intended to be within the scope of the claims which follow.

What is claimed is:

1. An endovascular embolization system comprising:
a braided implantable device comprising:
a tubular braid having an inner passageway defined longitudinally therein from a proximal edge to a distal edge; wherein the tubular braid is pre-formed as a default shape into multiple distinct sections including:
a stabilizing section;
a distal section including the distal edge;
a sealing section disposed proximally of the stabilizing section;
an intermediate section interposed directly between the stabilizing section and the sealing section; the intermediate section having a stiffness greater than that of the stabilizing section; and
a proximal section including the proximal edge;
wherein each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the distal section, the intermediate section, and the proximal section.

2. The system in accordance with claim 1, wherein the braided implantable device further comprises a coil having a free distal tip and a proximal end secured to the distal section of the tubular braid; wherein the coil is made of a shape memory material pre-formed to have an atraumatic coiled distal surface including a section of the coil proximally of the free distal tip of the coil.

3. The system in accordance with claim 2, wherein the distal edge of the distal section of the tubular braid and the proximal end of the coil are secured via a distal securement member; and in the pre-formed default shape the distal section of the tubular braid is offset at an acute angle relative to a longitudinal axis through the tubular braid.

4. The system in accordance with claim 1, further comprising:
a distal securement member fixedly secured about the distal section of the tubular braid; and
a proximal securement member fixedly secured about the proximal section of the tubular braid.

5. The system in accordance with claim 4, wherein the distal section including the distal edge is disposed distally or interiorly of the stabilizing section; and the proximal section including the proximal edge is disposed proximally or interiorly of the sealing section.

6. The system in accordance with claim 2, wherein at least a portion of the atraumatic coiled distal surface is substantially perpendicular to a longitudinal axis through the tubular braid.

7. A method for disrupting blood flow to a target site using an endovascular embolization system that includes a braided implantable device comprising: a tubular braid having an inner passageway defined longitudinally therein from a proximal edge to a distal edge; wherein the tubular braid is pre-formed as a default shape into multiple distinct sections including: a stabilizing section; a distal section including the distal edge; a sealing section disposed proximally of the stabilizing section; an intermediate section interposed directly between the stabilizing section and the sealing section; the intermediate section having a stiffness greater than that of the stabilizing section; and a proximal section including the proximal edge; wherein each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the distal section, the intermediate section, and the proximal section; the method comprising the steps of:
navigating a microcatheter through a vasculature to the target site;
while in the radially constricted state, pushing in a distal direction the braided implantable device through the microcatheter using a pushing member; and
upon exiting from a distal end of the microcatheter, deploying the braided implantable device at the target site; wherein when deployed the stabilizing section anchoring in position by radially expanding in direct physical contact with a vessel wall at the target site and together with the intermediate section pushing against and stabilizing in place at the target site the sealing section thereby minimizing risk of migration in a distal direction over time.

8. The method in accordance with claim 7 wherein the braided implantable device further comprises a coil having a free distal tip and a proximal end secured to the distal edge of the distal section of the tubular braid; wherein the coil is made of a shape memory material having a pre-formed default shape with an atraumatic coiled distal surface including a section of the coil proximally of the free distal tip of the coil; wherein during the step of pushing the braided implantable device through the microcatheter, the coil is in a longitudinally extended state wherein the deploying step further comprises the step of the coil automatically reverting to the pre-formed shape having the atraumatic coiled distal surface acting as a bumper preventing damage to the vessel wall during positioning at the target site while maximizing contact surface area with the vessel wall.

9. The method in accordance with claim 8, wherein at least a portion of the atraumatic coiled distal surface of the coil is substantially planar.

10. The method in accordance with claim 8, wherein the distal edge of the distal section of the tubular braid and the proximal end of the coil are secured via a distal securement member; and in the pre-formed default shape the distal section of the tubular braid is offset at an acute angle relative to a longitudinal axis through the tubular braid.

11. The method in accordance with claim 8, wherein at least a portion of the atraumatic coiled distal surface is substantially perpendicular to a longitudinal axis through the tubular braid.

12. The method in accordance with claim 7, wherein the braided implantable device further comprises:
a distal securement member fixedly secured about the distal section of the tubular braid;
a proximal securement member fixedly secured about the proximal section of the tubular braid.

13. The method in accordance with claim 12, wherein the distal section including the distal edge is disposed distally or interiorly of the stabilizing section; and the proximal section including the proximal edge is disposed proximally or interiorly of the sealing section.

14. The method in accordance with claim 7, wherein during the deploying step neither the distal section nor the proximal section expand radially.

15. A method of manufacture an endovascular embolization system, the method comprising the steps of:
providing a tubular braid having an inner passageway defined longitudinally therein from a proximal edge to a distal edge;
pre-forming the tubular braid as a default shape into multiple distinct sections including:
a stabilizing section;
a distal section including the distal edge;
a sealing section disposed proximally of the stabilizing section;
an intermediate section interposed directly between the stabilizing section and the sealing section; the intermediate section having a stiffness greater than that of the stabilizing section; and
a proximal section including the proximal edge;
wherein each of the stabilizing section and the sealing section are self-expanding between a radially expanded state when free from an externally applied radial force and a radially constricted state when subject to the externally applied radial force; each of the stabilizing section and the sealing section while in the radially expanded state have a maximum outer diameter larger than an outer diameter of each of the distal section, the intermediate section, and the proximal section.

16. The method in accordance with claim 15, further comprising securing about the distal section and the proximal section a distal securement member and a proximal securement member, respectively.

17. The method in accordance with claim 15, wherein after the pre-forming step and before the securing step further comprising heating to an inverted shape: (i) the distal section including the distal edge to be disposed interiorly of the stabilizing section; and/or (ii) the proximal section including the proximal edge to be disposed interiorly of the sealing section.

18. The method in accordance with claim 17, wherein the heating step further comprises heating a coil having a free distal tip and a proximal end; wherein the coil is made of a shape memory material pre-formed to have an atraumatic coiled distal surface including a section of the coil proximally of the free distal tip of the coil; and wherein the securing step comprises attaching via the distal securement member the distal section of the tubular braid and the proximal end of the coil.

19. The method in accordance with claim 18, further comprising shaping the distal section with the distal securement member secured thereabout at an acute angle offset relative to a longitudinal axis through the tubular braid while simultaneously creating the atraumatic distal surface.

20. The method in accordance with claim 18, wherein at least a portion of the atraumatic coiled distal surface is substantially perpendicular to a longitudinal axis through the tubular braid.
